(19) United States
(12) Patent Application Publication
(10) Pub. No.: US 2024/0175982 A1
(43) Pub. Date: May 30, 2024

(54) SYSTEMS AND METHODS FOR VEHICLE OCCUPANT CLASSIFICATION USING IN-CABIN SENSING

(71) Applicant: VEONEER US, LLC, Southfield, MI (US)

(72) Inventor: Shan Cong, Superior Township, MI (US)

(21) Appl. No.: 18/072,660

(22) Filed: Nov. 30, 2022

(51) Int. Cl. G01S 7/41; A61B 5/00; A61B 5/08

(52) U.S. Cl. CPC G01S 7/415; A61B 5/0816; A61B 5/6893; B60R 21/01512

(57) ABSTRACT

Methods and systems for location, identification, and/or classification of vehicle occupants using, at least in part, RADAR or other electromagnetic sensor data from within the vehicle cabin. In some implementations, the method may comprise identifying a vehicle occupant within a vehicle using electromagnetic signals and assigning the vehicle occupant to a location within the vehicle by processing electromagnetic signals. One or more features about the vehicle occupant may be extracted by processing electromagnetic signals and used to classify the occupant and/or alter a function of the vehicle.

### SUMMARY

Systems and methods are disclosed herein for using one or more sensors within the cabin of a vehicle, such as RADAR sensors, to identify, extract features from, and/or classify occupants within the vehicle. In some embodiments, changes within the vehicle may also be identified relevant to the location, identification, classification, and/or status of the vehicle occupants to improve upon the ability of the system to update and maintain signal processing as the scene within a vehicle changes.

In a more particular example of a method for identification and/or classification of a vehicle occupant, the method may comprise identifying a vehicle occupant within a vehicle using electromagnetic signals; assigning the vehicle occupant to a location within the vehicle by processing electromagnetic signals; and extracting one or more features about the vehicle occupant by processing electromagnetic signals.

Some implementations may further comprise classifying the vehicle occupant by processing electromagnetic signals.

In some implementations, the step of identifying a vehicle occupant may comprise detecting a vital sign of the vehicle occupant, such as a heart rate or breathing rate, for example.

In some implementations in which a vital sign is detected, this may be accomplished by identifying a repeating pattern of Doppler spectrum peaks in a RADAR signal using at least one range bin and identifying an estimated frequency distance between adjacent peaks of the repeating pattern. An estimated breathing rate or another vital sign may be calculated using the estimated frequency distance.

Some implementations may further comprise identifying a scene change within the vehicle. For example, a change in a number of vehicle occupants within the vehicle may be identified, in some cases using electromagnetic signals. Alternatively, or additionally, a change in a location of vehicle occupants within the vehicle may be identified, again preferably using electromagnetic signals. Other scene change may be identified by, for example, identifying movement in a door of the vehicle and/or identifying a threshold change in velocity of the vehicle.

Some implementations may further comprise, in response to identifying the scene change, changing at least one processing parameter associated with at least one of the steps of: (1) extracting one or more features about the vehicle occupant; and (2) classifying the vehicle occupant, such as resetting at least one variable used during the step of classifying the vehicle occupant. For example, a buffer associated with one or more of the seats/vehicle occupants may be reset.

In an example of a method for classification of an object within a vehicle using RADAR signal processing, the method may comprise identifying an object, such as preferably a human or other living vehicle occupant, within a vehicle using RADAR signals. The object may be assigned to a location within the vehicle by processing RADAR signals. One or more features about the object may be extracted by processing RADAR signals. The object may then be classified by processing RADAR signals.

Some implementations may further comprise identifying a scene change within the vehicle. In some such implementations, in response to identifying the scene change, one or more processing parameters of the vehicle may be changed, such as resetting a cache or buffer, for example. In some implementations, identification of a scene change may take place as an alternative to classifying the object by processing RADAR signals.

In some implementations in which the object comprises a living being and/or animal, such as a human, the step of extracting one or more features about the object by processing RADAR signals may comprise estimating a rate associated with a vital sign of the human within the vehicle. In some such implementations, the step of extracting one or more features about the object by processing RADAR signals may comprise calculating a variance of a frequency difference between Doppler peaks associated with the RADAR signals.

In a more particular example of a step of extracting one or more features about the object by processing RADAR signals, this step may comprise identifying a repeating pattern of Doppler spectrum peaks in a RADAR signal using one or more range bins; identifying an estimated frequency distance between adjacent peaks of the repeating pattern; and calculating an estimated rate of a repeating vital sign of the occupant within a cabin of the vehicle using the estimated frequency distance.

In some implementations, the step of identifying an object may comprise identifying the object as a human, which, in some such implementations, may comprise identifying a vital sign associated with the object. In some such implementations, the step may further comprise recognizing the vital sign as being that of a human or, alternatively, of another animal, such as a dog, cat, or other pet.

In example of a system for classification of a vehicle occupant using electromagnetic signal processing according to some embodiments, the system may comprise one or more electromagnetic sensors positioned within a cabin of a vehicle, such as one or more RADAR sensors. The system may further comprise a location detection module configured to process reflected electromagnetic signals to estimate a location of a vehicle occupant within the cabin of the vehicle. A feature extraction module of the system may be configured to extract one or more features about the vehicle occupant by processing reflected electromagnetic signals.

Some embodiments may further comprise a classification module configured to classify the vehicle occupant using features extracted using the feature extraction module. In some such embodiments, the classification module may be configured to classify the vehicle occupant according to an estimated age group using a rate associated with a vital sign of the vehicle occupant obtained from the electromagnetic sensor.

In some embodiments, the feature extraction module may be configured to identify a vital sign and/or vital sign rate, such as a breathing rate or a heart rate, for example, associated with the vehicle occupant. In some such embodiments, the feature extraction module may be configured to use a Doppler signal repetition frequency to estimate a rate associated with a vital sign of the vehicle occupant.

Some embodiments may be configured to classify the vehicle occupant according to an estimated age group using a rate associated with a vital sign of the vehicle occupant obtained from the electromagnetic sensor.

The features, structures, steps, or characteristics disclosed herein in connection with one embodiment may be combined in any suitable manner in one or more alternative embodiments.

### BRIEF DESCRIPTION OF THE DRAWINGS

Non-limiting and non-exhaustive embodiments of the disclosure are described, including various embodiments of the disclosure with reference to the figures, in which:

FIG. 1 depicts a vehicle comprising a system for detection of vehicle occupant vital signs according to some embodiments;

FIG. 2 is a graph representing Doppler spectrum signals received from a vehicle occupant;

FIG. 3 is a graph representing a series of signals, each in a different range bin, representing data from which a vehicle occupant vital sign may be derived;

FIG. 4 is a chart illustrating a statistical correlation between age and breathing rates in breaths per minute that may be used to classify vehicle occupants according to a vital sign;

FIG. 5 is a flow chart depicting a method for classification of an occupant using feature data according to some implementations;

FIGS. 6A-6C depict schematic diagrams representative of an example of a system for in-cabin vehicle occupant classification and/or feature extraction according to some embodiments;

FIG. 7 depicts an example of a system for location assignment of a vehicle occupant or other target within a vehicle and/or classification using such location assignment(s) according to some embodiments;

FIG. 8 depicts an example of a system for detection and/or processing of scene change data within a vehicle using, at least in part, RADAR or other electromagnetic signals according to some embodiments;

FIG. 9 depicts an example of a system for occupant classification within a vehicle according to some embodiments;

FIG. 10 depicts an example of a system for processing of vehicle occupant features and/or classification of occupants in a vehicle according to some embodiments; and

FIG. 11 is a flow chart depicting an example of a method for classification of vehicle occupants according to some implementations.

### DETAILED DESCRIPTION

It will be readily understood that the components of the present disclosure, as generally described and illustrated in the drawings herein, could be arranged and designed in a wide variety of different configurations. Thus, the following more detailed description of the embodiments of the apparatus is not intended to limit the scope of the disclosure but is merely representative of possible embodiments of the disclosure. In some cases, well-known structures, materials, or operations are not shown or described in detail.

As used herein, the term “substantially” refers to the complete or nearly complete extent or degree of an action, characteristic, property, state, structure, item, or result to function as indicated. For example, an object that is “substantially” cylindrical or “substantially” perpendicular would mean that the object/feature is either cylindrical/perpendicular or nearly cylindrical/perpendicular so as to result in the same or nearly the same function. The exact allowable degree of deviation provided by this term may depend on the specific context. The use of “substantially” is equally applicable when used in a negative connotation to refer to the complete or near complete lack of an action, characteristic, property, state, structure, item, or result. For example, structure which is “substantially free of” a bottom would either completely lack a bottom or so nearly completely lack a bottom that the effect would be effectively the same as if it completely lacked a bottom.

Similarly, as used herein, the term “about” is used to provide flexibility to a numerical range endpoint by providing that a given value may be “a little above” or “a little below” the endpoint while still accomplishing the function associated with the range.

The embodiments of the disclosure may be best understood by reference to the drawings, wherein like parts may be designated by like numerals. It will be readily understood that the components of the disclosed embodiments, as generally described and illustrated in the figures herein, could be arranged and designed in a wide variety of different configurations. Thus, the following detailed description of the embodiments of the apparatus and methods of the disclosure is not intended to limit the scope of the disclosure, as claimed, but is merely representative of possible embodiments of the disclosure. In addition, the steps of a method do not necessarily need to be executed in any specific order, or even sequentially, nor need the steps be executed only once, unless otherwise specified. Additional details regarding certain preferred embodiments and implementations will now be described in greater detail with reference to the accompanying drawings.

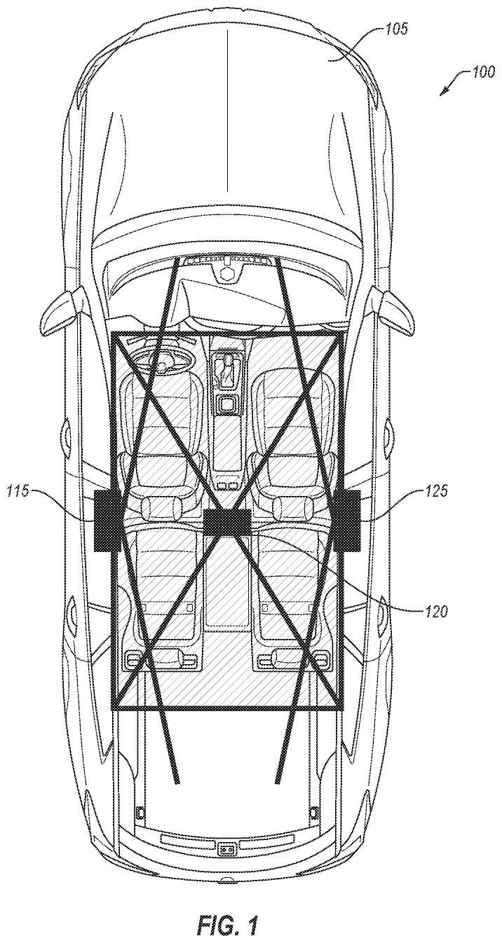

FIG. 1 depicts a system 100 for detection and/or classification of vehicle occupants within the cabin of a vehicle 105. As shown in this figure, one or more sensors may be positioned at various locations within the cabin of vehicle 105. In the depicted embodiment, vehicle 105 comprises three sensors 115, 120, and 125. Sensor 115 is positioned on one side of the vehicle 105, sensor 120 is positioned at a central location within vehicle 105, such as mounted on the ceiling of the cabin, and sensor 125 is mounted on the opposite side of vehicle 105. As those of ordinary skill in the art will appreciate, however, a wide variety of alternatives are possible, including different numbers of sensors, different types of sensors, and different locations of sensors.

For example, in preferred embodiments, sensors 115, 120, and 125 may comprise RADAR sensors, such as a frequency modulated continuous wave (FMCW) ultra-wide band RADAR sensor configured to operate at 60 GHz. However, in alternative embodiments, other types of sensors may be used, such as LIDAR or other types of electromagnetic sensors, for example. In addition, in some embodiments, a single sensor may be used, such as sensor 120, or two sensors (one in the middle and one on just one side of the vehicle, for example). More than three sensors may also be used in some embodiments. Although it may be preferably to locate the sensors in the roof/ceiling or the upper side of a vehicle pillar, in some such embodiments, or in alternative embodiments having three or fewer sensors, such sensor(s) may alternatively be positioned in the front of the vehicle, the rear of the vehicle, within seats of the vehicle, and/or in the floor of the vehicle, for example.

As illustrated by the various lines extending from sensors 115/120/125, each of these sensors may be configured to direct electromagnetic signals to and/or receive electromagnetic signals from particular regions of the cabin of vehicle 105, preferably so as to at least be capable of detecting occupants within each of the seats of the vehicle 105. Of course, again, many alternatives are contemplated and/or would be available to those of ordinary skill in the art after having received the benefit of this disclosure. For example, a single sensor positioned at a suitable location may, for some vehicles, be sufficient to adequately detect occupants in every seat in the vehicle. Similarly, in other embodiments, it may be desirable to provide a dedicated RADAR or other electromagnetic sensor for each seat of the vehicle.

As described in greater detail below, irrespective of the placement, number, and type of electromagnetic sensors used in the vehicle, in preferred embodiments, such sensor(s) may be used to identify one or more occupants present in the cabin and, in some embodiments, identify features, such as vital signs, about such occupant(s). As described in greater detail below, some embodiments may further be configured to use signal data and/or features from the signal data, such as vital signs, to classify the vehicle occupants, which may allow for modification of other parameters of the vehicle and/or otherwise taking actions in the vehicle in response thereto.

Feature data, such as vital sign data—which may include breathing rates, tidal volume changes, and/or heart rates, for example—range/height data, range extent data, and the like, may be used to classify the occupant(s). For example, in some embodiments, vehicle occupants may be classified using vital sign data according to their age group. By doing so, various actions may be taken and/or vehicle functions changed based upon the classification and/or location of the vehicle occupant(s).

Vital sign data may be collected, for example, using RADAR frequency response data from vital signals of the occupant(s). To provide an example of a model for tracking vital sign signals, consider the following equations:
s(t)=A(t)e^{jϕ(t)}; A(t) is the target amplitude

$$ϕ ( t ) = 2 p \frac{{r}_{0} + r ( t )}{λ} + d ( t ) ;$$

r₀ is the target nominal range.
r(t)=b sin ω0t; r(t) is the target motion over time and b is the maximum motion of the target
δ(t): phase noise

The frequency domain representation of the vital sign signal can then be represented as follows:

$$f ( ω ) = aT {\sum }_{n = - ∞}^{∞} {J}_{- n} ( β ) sin c ( ω - n {ω}_{0} ) \frac{T}{2}$$

where J₋ₙ is Besser's first integral and

$$β = - \frac{2 π b}{λ}$$

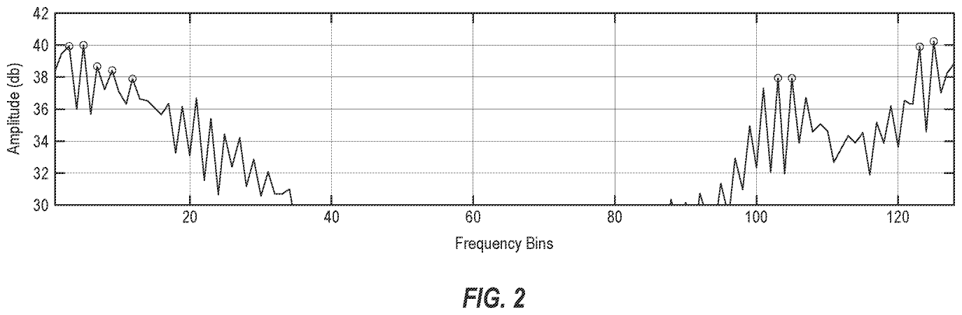

FIG. 2 is a graph representing Doppler spectrum signals received from an adult occupant of a vehicle. In the example provided in the figure, the adult is estimated to have a breathing rate of about 10.15 breaths per minute. As shown in this graph, using the equations above, the breathing rate can be derived from the distance between peaks in the signal or “repetition frequency,” several of which are circled in the graph.

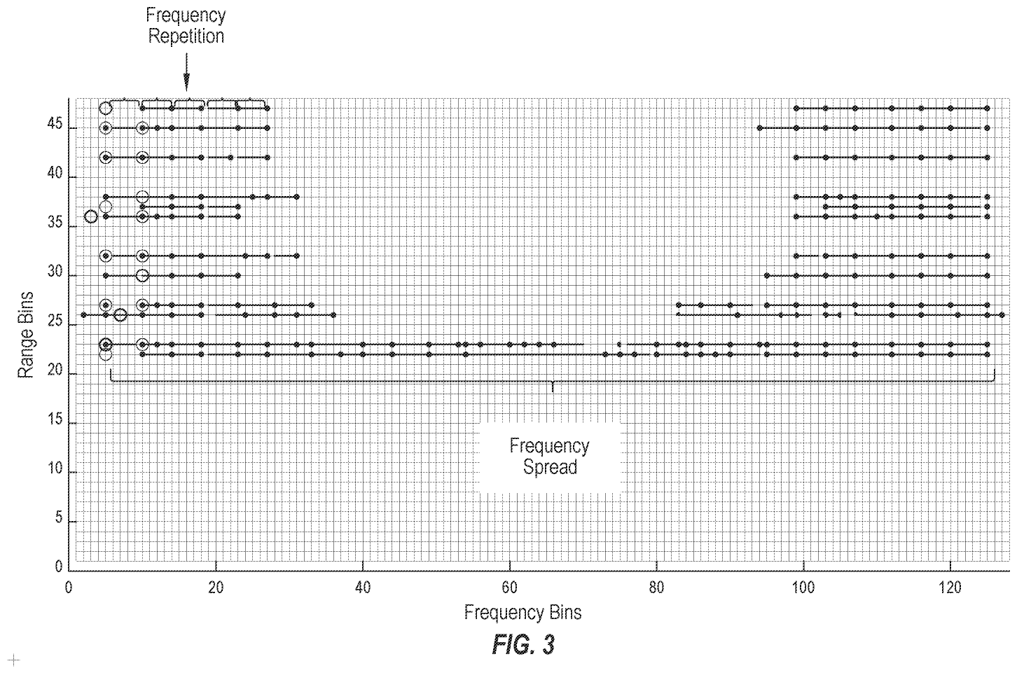

FIG. 3 is another graph representing an exemplar processed RADAR signal in range-Doppler format. Each range bin in this specific example represents a fixed distance or range of distances from the sensor, which is proportional to RADAR range resolution, and each frequency bin represents a fixed frequency band proportional to Doppler resolution. The signal shown on the graph of FIG. 2 is represented at range bin 22 in the graph of FIG. 3, which is the strongest signal detected among those shown in FIG. 3.

The Doppler spectrum peaks are represented by dots in the graph of FIG. 3 and, once again, some of these peaks are circled. The frequency repetition, which is represented by the brackets adjacent to range bin 47, can again be derived from the frequency spread shown in FIG. 3. Note that the signals from other range bins generally have the same frequency repetition rate, which may be attributed to other regions of the same occupant's body or from multipath propagation. Of course, some signals may be distinguished as coming from a different occupant or other object within the vehicle. As those of ordinary skill in the art will appreciate, such signals may be processed and/or extracted to be identified as such by way of processing and/or filtering steps disclosed herein or otherwise available to those of ordinary skill in the art.

As mentioned above, although the data shown in FIGS. 2 and 3 represent breathing rates, other vital signs may be estimated using similar techniques. For example, estimated heart rates and/or tidal volume changes may alternatively be derived from RADAR or other electromagnetic signals using the methods disclosed herein.

Once a breathing rate or other vital sign has been estimated for one or more occupants with a vehicle using RADAR or other electromagnetic signal processing, this vital sign data may be used to classify the occupant(s). For example, because respiratory rates are strongly correlated with age, an estimate of the age of the occupant may be made using the estimated breathing rate derived from the RADAR signal processing. As described in greater detail below, other features may be derived from reflected or otherwise received RADAR or other electromagnetic signal data, such as location, range/height data, range extent data, and the like, in various contemplated embodiments.

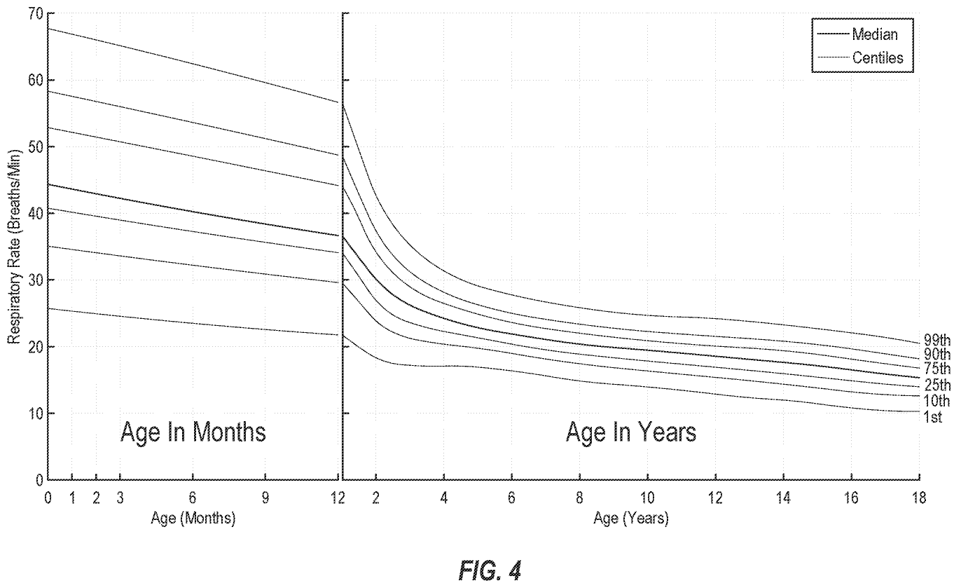

FIG. 4 is a chart illustrating the statistical correlation between age and breathing rates in breaths per minute. This chart illustrates how, for example, a breathing rate of about 40 breaths per minute might reasonably be used to classify an occupant as an infant. Thus, using statistical data, such as from the chart of FIG. 4 or other similar data, a vehicle may be configured to use breathing rates, or other vital sign rates, to classify an occupant according to the occupant's predicted age group. As described in greater detail below, this classification may then be used in various ways by the vehicle to perform useful functions.

For example, if an occupant is identified as being an infant, an airbag may be automatically disabled in the seat associated with this occupant. As another example, if an occupant is identified as an infant or child and remains in the vehicle after the vehicle has been turned off and/or the driver of the vehicle has exited the cabin, the vehicle may be configured to send notifications or warnings regarding the occupant left behind. In some embodiments, the vehicle may be configured to continue to monitor the occupant(s) remaining in the vehicle and only provide such notifications in the event of a detected confluence of data, which may include data from other sensors.

For example, if the vital sign data from the RADAR or other electromagnetic sensor has classified the occupant as an infant or child, rather than immediately sending a notification, the vehicle may be configured to monitor other data, such as temperature and/or time since the child has been left within the vehicle. Upon detecting a temperature within the cabin beyond a threshold temperature, such as 90 degrees Fahrenheit, for example, in combination with data indicative of a child being left in the vehicle, the vehicle may be configured to send a notification to a user. This warning may, for example, be sent to a smart phone of an owner/user of the vehicle. Alternatively, the vehicle may be configured to automatically start the engine and/or start an air conditioning unit of the vehicle in response to a detection of these triggers/thresholds. It should be understood that any of sensors 115/120/125 may therefore comprise a temperature sensor in some embodiments, which may operate in conjunction with RADAR or other electromagnetic sensors to achieve this result.

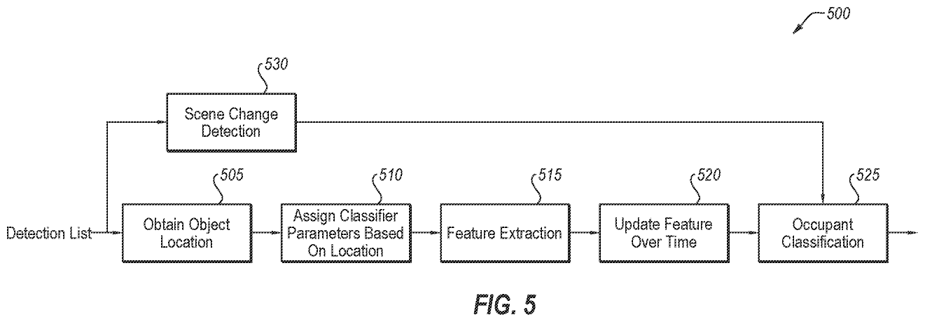

FIG. 5 is a flow chart depicting various steps involved in a method 500 for classification of an occupant using feature data according to some implementations. As shown in this figure, data from a detection data set, such as a list or other detection data set from RADAR signal processing, for example, may be received. In some implementations, this data may be received, for example, by a software module of a system of a vehicle, such as a vehicle occupant classification system.

The data from the detection list/data set may be used to obtain location information about an object within the vehicle, such as a vehicle occupant. In some implementations, and related embodiments, data used to obtain location information may comprise, for example, distance/range data, directional/bearing data, such as data from a directional antenna, and the like.

This data may be correlated with other data to obtain the location, or at least approximate location, of the occupant or other object within the vehicle at 505 by, for example, identifying each of the one or more objects/occupants in the vehicle as being included in a particular zone of the vehicle. For example, in some implementations and related embodiments, each of one or more objects/occupants may be identified as being in a particular row of the vehicle, such as front row, rear row, middle row, etc. In some implementations and embodiments, the objects/occupants may also be identified based upon a lateral zone of the vehicle, such as the left side or right side of the vehicle.

In some cases, the identification zones may each correspond with a single seat of the vehicle. In this manner, each occupant of the vehicle may be assigned to a specific seat. However, this need not be the case for all contemplated embodiments and implementations. For example, it may suffice for certain applications to simply assign a vehicle occupant to a particular row of the vehicle without regard to the lateral position of the occupant.

As described in greater detail below, in some embodiments and implementations, a location may be linked with a particular target by identifying one or more signals or detections from a detection list or data set having the largest signal-to-noise (SNR) ratio. Other signals/detections indicative of high location quality from the list/data set, such as those close to a signal/detection having the maximum SNR or those having a similar range, may be selected for further location processing.

The target location may be estimated using SNR weighted average of the X and Y coordinates of the selected signals/detections. In some implementations and embodiments, one or more coordinate/variable estimates may be compensated for under certain circumstances. For example, if one or more detections/signals have a statistically strong tendency towards one direction, such as a significant number of “X” coordinate values that fall in a particular side/region of the vehicle, the X coordinate may be compensated.

The coordinate estimates, such as “X” and “Y” coordinate estimates for a current frame, may then be stored, such as stored in a buffer. Various detection ratios may then be calculated, which may be used to assign a target to a position or region within the vehicle. For example, in some implementations and embodiments, the front footwell ratio, front ratio, and rear ratio may be calculated using values in the buffer. If, for example, most of the values in the x-coordinate buffer are in the x-range of the rear row of the vehicle, then the rear ratio will be high and the system/method may assign, or be more inclined to assign, the target to the rear row of the vehicle.

Similar techniques may be used to estimate and/or assign a target to a location/zone in another dimension. For example, in some cases based upon various ratios that may be calculated using signal processing, data from the y-coordinate buffer may be used to estimate and/or assign the target's position in terms of its lateral position.

Once the location or zone, with whatever specificity is most useful, has been obtained, one or more classifier parameters may be assigned at 510. These parameters may, for example, define the range and/or thresholds of feature values for each class.

In some implementations, one or more of these parameters may be assigned based, at least in part, upon the location identified at 505. For example, in some implementations and embodiments, each location/zone in the vehicle may have its own set of parameters used to classify vehicle occupants.

Of course, this need not be the case in all contemplated embodiments. For example, some embodiments and implementations may assign classifier parameters independent of location. As another example, some embodiments and implementations may assign classifier parameters that depend upon location, but may be identical for all but one, for example, or all but a subset of the locations/zones within the vehicle.

One or more features may then be extracted from the data at 515. Further details regarding such feature extraction can be found below, but examples of features that may be extracted from RADAR or other electromagnetic signal data from within the cabin of a vehicle include variance of frequency-difference between Doppler peaks, spread bandwidth, number of valid detections, number of valid Doppler spectrum peaks, range, range extent, and occupant vital signs, such as breathing rate and heart rate.

Features extracted in step 515 may be updated over time as desired at 520. For example, some features may be updated continually over time. Other features may only be updated in certain circumstances, such as upon detection of a scene change, as indicated at step 530, for example.

The target/occupant may then be classified at step 525. For example, in some embodiments and implementations, step 525 may comprise classifying one or more vehicle occupants according to their predicted age or age group. For example, a vehicle occupant may be classified as an adult, child, or infant based upon a vital sign being within a particular range and/or beyond a certain threshold. As explained below, this classification data may then be used in various other ways to improve upon vehicle occupant safety, for example.

As briefly mentioned above, some implementations and embodiments may be configured to detect various “scene changes” at 530. These scene changes may be used to alter one or more other data processing steps. For example, upon detecting that a vehicle door has opened and/or closed, an inference may be made that one or more vehicle occupants may have exited and/or entered the vehicle. Other examples of scene changes that may be detected and/or used in one or more embodiments include threshold changes in vehicle velocity, vehicle on/off switches, and/or detection-based scene changes, such as scene changes based upon a significant change in the number and/or type of signal detections within a vehicle, or a part of the vehicle. Such scene changes may then trigger a reset of one or more occupant classifications or an increase in the number of signals and/or data processing steps involved in one or more of the steps of method 500, such as any of the feature extraction 515, feature updating 520, and/or occupant classification 525 steps.

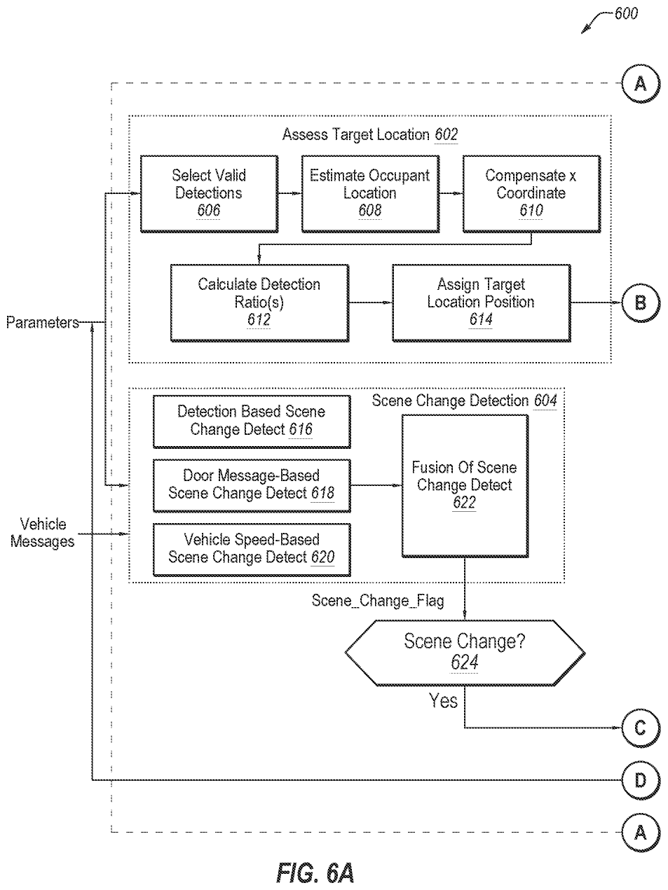

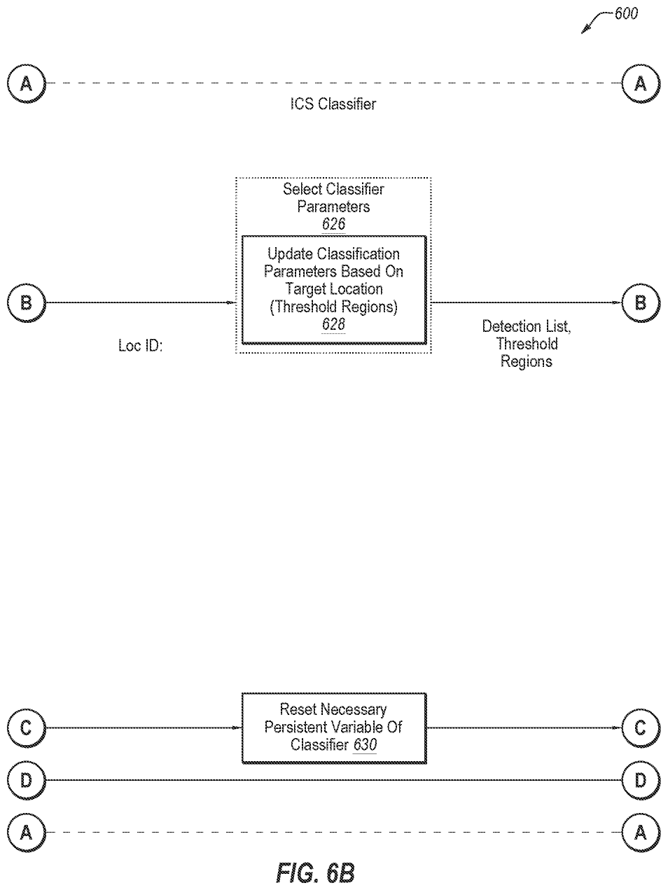

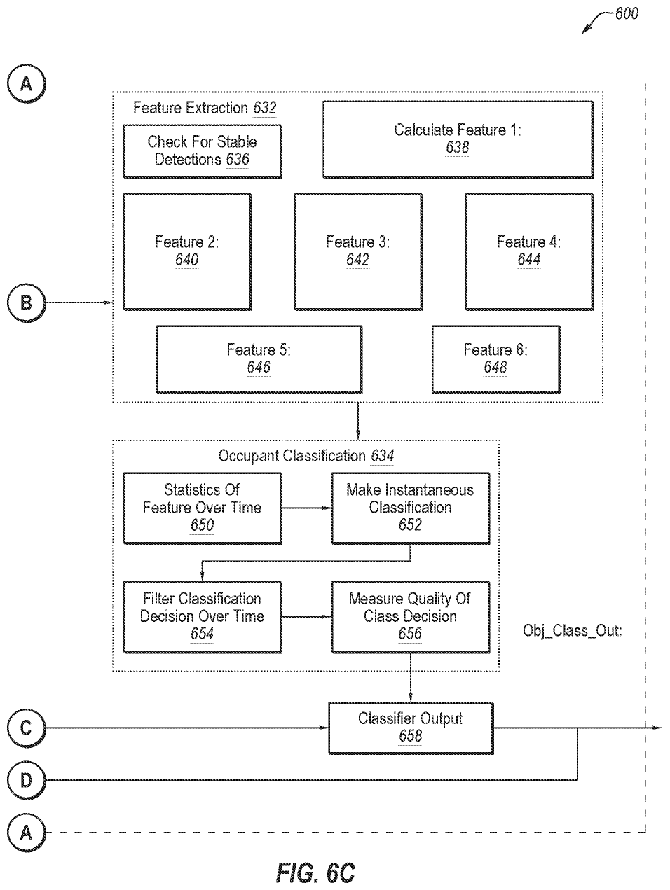

FIGS. 6A-6C depict a schematic diagram of a system 600 for in-cabin vehicle occupant classification and/or feature extraction according to some embodiments. A location/zone of one or more targets identified by RADAR or other electromagnetic signals may be assessed at 602. Parameters may be used at 606 to select one or more signal detections, such as one or more signal detections identified as valid or otherwise useful for further processing. Examples of parameters for this purpose include parameters relating to signal detections, vital signs, and/or location data of the detected objects/targets.

In some embodiments, selection of valid detections may comprise identifying signals with a maximum and/or threshold SNR. In some embodiments, additional detections that are sufficiently close to the maximum SNR detection and/or the range of the detections having the maximum and/or threshold SNR may be selected for further location processing.

An estimate of a location of one or more vehicle occupants may then be made at 608. As previously mentioned, this estimate may be made to assign each target/occupant with a particular seat in the vehicle. Alternatively, the estimate may be less refined, and may, for example, assign a target to a particular row, to a front/back of the vehicle, and/or left/right lateral side of the vehicle. In some embodiments, the target location may be estimated using, for example an SNR weighted average of one or more dimensional coordinates of each of the selected detections, such as the “X” and “Y” coordinates, which may correspond to the front/rear and lateral side to side coordinates of the vehicle.

In some embodiments, one or more of the variables/coordinates, such as the X coordinate, may be compensated at 610. In some embodiments, this compensation may be based upon whether a significant number, such as a threshold number and/or percentage, of the X-coordinate (using the X coordinate example) values fall within a particular side/range/threshold, such as in the front side/rear side of the vehicle, to avoid dithering of the location assignment. This step may be useful if a significant number of detections have a statistically strong correlation with one side/direction/location/dimension of the vehicle.

One or more detection ratios may then be calculated at 612. For example, in some embodiments, the estimates for one or more coordinates, such as “x” & “y” coordinate estimates, of a current frame in a buffer may be calculated. In some such embodiments, one or more ratios may be calculated using such data, such as, for example, the front footwell ratio, front ratio, and/or rear ratio, based on the values in the buffer. For example, if most of the values in the x-coordinate buffer are in the x-range of the rear row of the vehicle, then the rear ratio will be high, which may indicate a likelihood of the target being in the rear row.

The processed location data may then be used to assign the target to a particular seat, location, and/or zone at 614. For example, in some embodiments, the ratio(s) calculated in step 612 may be used to assign the target to a particular row of the vehicle. In some embodiments, this row assignment may be combined with similar data used to assign the target's position further, such as in terms of left to right lateral position and/or to a particular seat in the vehicle. This target location assignment data may then be saved and used for further actions/processing.

Some embodiments may also, or alternatively, be configured to detect changes within the cabin of the vehicle that are or may be relevant to identification and/or classification of occupants within the vehicle—or, as used herein, “scene changes” within the vehicle—as indicated at 604. Such scene change detection may, in some embodiments, comprise receipt of messages and/or data from the vehicle itself, such as data indicating that the vehicle has been turned on, turned off, and/or one or more doors of the vehicle have been opened and/or closed. As indicated in FIG. 6A, some embodiments may further conduct scene change detection using one or more parameters (in some cases the same parameter data used to assess the target location at 602).

Various types of data may be, in some embodiments, fused or otherwise combined in order to detect a scene change at 622. For example, the depicted embodiment is configured to use detection-based scene change data 616, door and/or vehicle message-based scene change data 618, and vehicle speed/velocity-based scene change data 620.

Detection-based scene change data 616 may comprise, for example, a threshold number or percentage change in the number of detections associated with the vehicle, or a particular part of the vehicle. In some embodiments, the types of detections and/or the values for certain detections in the vehicle, or a particular part of the vehicle, may be indicative of a scene change. Detection-based scene change data 616 may also, in some embodiments, comprise locational data and/or vital sign data, which may be used to detect threshold movement in a cabin.

A door/vehicle-message based scene change 618 may be triggered upon detecting that one or more doors have been opened and/or closed. In some embodiments, detection of any opening and/or closing of a vehicle door may be treated identically in terms of scene change processing. Alternatively, such detections associated with one or more particular doors may be processed differently. For example, in some embodiments, a detection of an opening and/or shutting of the driver's door may trigger unique data processing steps and/or actions.

A vehicle speed/velocity-based scene change 620 may be triggered, for example, when the vehicle speed changes beyond a certain threshold and/or beyond a certain period of time. For example, if a vehicle speed changes from being at rest for a threshold period of time to moving, the vehicle may be configured to indicate a scene change that is associated with predicted stability and therefore requires less signal processing during this “scene” or status. Similarly, if a vehicle speed changes to zero, in some cases for a threshold period of time, a scene change may be detected that is associated with additional signal processing, since it may be expected that one or more vehicle occupants may have changed during this scene change.

After processing of the scene change data at 622, a decision may be made at 624 as to whether a scene change, or a scene change of threshold significance, has taken place or likely taken place. In some embodiments, certain types of scene change data, such as data indicative of a door opening and/or closing, for example, may automatically trigger a scene change at 624. However, other types of scene change data may require processing and/or analysis to determine whether a scene change has taken place. For example, confirmation of a scene change may require the confluence of speed change data, such as speed change beyond a threshold or any change to or from zero, with time data, which may prevent stopping at a traffic light, for example, from automatically triggering a scene change.

The assigned location data or Loc ID, may be used to select classifier parameters at 626, as shown in FIG. 6B. In some embodiments, one or more classification parameters may be selected based at least in part on the target object location and whether the target object class is already known. Such parameters may then, in some embodiments, be used in a detection feature extraction module to check whether the estimated feature values fall within one or more predefined threshold regions of the classes.

In embodiments in which the classification parameters are based upon target locations, as indicated at 628, some such embodiments may involve, for each location within the vehicle, selection of the parameters for classification, which parameters may be used to define the range (upper and lower bounds) or thresholds (simply an upper or lower bound) of feature values for each class. By basing these selections on location, some locations and/or features may be defined more precisely than others. For example, a location corresponding with a driver of the vehicle may have ranges and/or thresholds defined with greater precision and/or with more detections required in order to increase the likelihood of an accurate classification.

If the classification for a particular target has matured (‘class_status’=1), the current known class (adult/child/baby, for example) and target location may also be used in the selection of classification parameters to help the consistency of the class decision.

As also shown in FIG. 6B, upon confirmation of a suspected scene change (see FIG. 6A), one or more variables of the classifier may be reset at 630, indicating that buffered/previous data may not be useful for one or more locations/seats, or for the entire vehicle, moving forward. If the scene change cannot be confirmed at 624, the features, variables, and/or classifications may continue to be updated over time rather than reset. Some embodiments may comprise resetting data, such as buffers, caches, etc., associated with the entire scene. Alternatively, some embodiments may comprise resetting only data associated with a particular seat or region of the vehicle, as may be suggested by the scene change data.

As shown in FIG. 6C, the data from 626/628, which may comprise a detection list, various thresholds/ranges, and/or target location data, may be used for feature extraction at 632. This process may be used to estimate the classification feature values for current detection information. Examples of such feature values involved in preferred embodiments include variance of frequency-difference between Doppler peaks, spread bandwidth, number of valid detections, number of valid Doppler spectrum peaks, range, range extent, and vital sign data.

The processing involved in feature extraction may comprise identification of stable and/or valid detections in the vehicle cabin, as indicated at 636. In some embodiments, the history and/or quality of detections may be considered in finding stable detections. One or more features may then be estimated/calculated using the incoming data. In the depicted embodiment, six different features, namely features 638, 640, 642, 644, 646, and 648 are used, although those of ordinary skill in the art will appreciate that any number of features, more or less than shown in the example, may be used.

As a specific example of useful features that may be used to classify vehicle occupants, one of the features may comprise the variance of frequency difference between Doppler peaks associated with the stable detections in one or more locations within the vehicle. This data may provide useful information about the stability/movement of the target and/or targets associated with the target location. In some embodiments, the processing may involve calculation of the weighted frequency-difference variance, wherein the weights correspond to the amplitude of the peaks in the signals.

For example, the variance feature may be calculated as Var=√Σ̅ᵢ̅^̅{̅N̅}̅w̅ᵢ̅(̅f̅ᵢ̅^̅{̅d̅i̅f̅f̅}̅−̅μ̅ᵢ̅^̅{̅d̅i̅f̅f̅}̅)̅²̅ where N is the number of valid peaks, wᵢ is the normalized weights related to the Doppler frequency peak amplitude, fᵢ^{diff} is the frequency-difference between i^{th} peak and its adjacent peak, and μᵢ^{diff}=Σᵢ^{N} wᵢ fᵢ^{diff} is the weighted mean of all fᵢ^{diff}.

As another specific example of useful features that may be used to classify vehicle occupants, one of the features may comprise the number of valid Doppler spectrum peaks in the data set. The total number of valid Doppler spectrum peaks may be calculated as Σᵢ^{N} nᵢ where N is the number of valid detections, nᵢ is the number of peaks in the Doppler spectrum for the i^{th} detection.

As mentioned above, the number of valid detections may be used as another feature to be extracted from the data. This may, for example, represent the number of valid detections available in the current frame. Some objects/targets may generate large number of detections (larger objects) while others may generate lesser detections (smaller objects). Thus, the number of detections may be used to extract data about the size of the target object.

Other potential feature may comprise use of range data, such as minimum range detections and/or range extent data. In some such embodiments, range data may be used to estimate a height and/or depth of a target occupant/object. If the range is the radial distance of the target from the RADAR or other sensor, the minimum and maximum range values for the valid detections may be calculated as: max([r₁r₂ . . . rᵢ . . . r_{N}]) and min([r₁r₂ . . . rᵢ . . . r_{N}]) where N is the number of valid detection and rᵢ is the range of the i^{th} detection. In some embodiments, the range may be directly related to the height of an object at a specified location.

Range extent may also, or alternatively, be used as another feature. The range extent may be the difference between the maximum and minimum range values for the valid detections associated with a target and/or target location. Small objects tend to have small range extent, while larger objects typically have larger range extent. Thus, the range extent may also provide useful data for classification and/or other actions, processing or otherwise, within the vehicle. Range extent may be calculated as follows: max([r₁r₂ . . . rᵢ . . . r_{N}])−min([r₁r₂ . . . rᵢ . . . r_{N}]) where N is the number of valid detection and nᵢ is the range of the i^{th} detection.

Yet another potentially useful feature that may be used in various embodiments comprises the spread bandwidth. The spread bandwidth may be calculated as Σᵢ^{N} SBᵢ where N is the number of valid detection with unique range and SBᵢ is the maximum Doppler bandwidth for the detection at the i^{th} unique range. The spread bandwidth may be used as a proxy/indicator for tidal volume of breathing, which is a combination of body displacement and speed of chest motion, and may therefore be used to derive a breathing rate and/or size of a target occupant.

Still another example of a feature that may be extracted from the RADAR or other electromagnetic signal data within the vehicle cabin comprises vital sign data, such as breathing rate, heart rate, or other vital sign data. This data may be used to distinguish living occupants, such as humans or pets, from non-living objects in a vehicle cabin and, in some cases, may be used to classify the living occupant, such as according to a particular age or age group.

For example, in some embodiments, the system may be configured to identify a target signal, such as a target range bin or other data set associated with the target and/or target range, from a detection list and/or data set. In some embodiments and implementations of related methods, this may be done by identifying the strongest signal and/or range bin signal from a list of detections available in the detection structure/data set and having a repeating pattern indicative of a vital sign. Thus, referring back to the chart of FIG. 3, which may be considered an example of a “detection list,” the detections in range bin 22 may be identified as the target signal or target bin.

This target signal data, including data gathered and/or processed prior to identification of the target signal/target bin and/or data gathered and/or processed following such identification, may then be used to obtain and refine a Doppler frequency spread spectrum. Thus, for example, the target signal data may be used to identify Doppler spectrum peak locations.

In some embodiments and implementations, a fast Fourier transform (FFT) methodology may be used to process the data and output an estimated rate associated with a vital sign. Thus, a buffered range of FFT data may be provided. In some such embodiments and implementations, the buffered range FFT data may be of the past N frames—for example, N may be equal to 128 frames). In some cases, this data may be associated with a particular target location corresponding with the vehicle occupant the vital sign of which is being monitored. Thus, multiple sets of data may be used, each corresponding with a different occupant. Alternatively, of course, some embodiments may be configured to simply detect the presence of a vehicle occupant without regard to the occupant's location.

In some embodiments and implementations, Doppler FFT may be performed. This may be performed for the target range bin/data set and, in some preferred embodiments, may also be performed for one or more range bins/data sets adjacent to the target range bin/data set or otherwise sufficiently related to the target range bin/data set to improve the vital sign estimation. For example, in some embodiments, each target range bin/data set within a particular number of bins of the target range bin may be used. Alternatively, each data set within a threshold signal strength of the target range bin/data may be used.

In some embodiments and implementations, cross-channel processing may also be performed, preferably over all received signal channels associated with the target range bin/data set and/or one or more adjacent or otherwise sufficiently related to the target range bin/data set. This may be accomplished, for example, by performing averaging and/or cross-channel processing over all received channel signals. Alternatively, this may be accomplished by beamforming the signals to an intended direction, which may be determined following location of the occupant within the vehicle and/or initial signal processing to determine a more precise location associated with a particular occupant to improve signal strength, such as a particular part of the occupant's chest for breathing rate estimation or the occupant's heart for heart rate estimation.

Data from cross-channel processing may then be used to estimate the current vital sign, such as the current breathing rate. In some embodiments and implementations, this may be achieved by determining frequency peak distances in a set of signal data associated with the target range bin(s)/target data set. Thus, for example, Doppler spectrum peaks in the target range bin(s) of interest and/or target signal data set may be identified and/or stored. The Doppler spectrum peaks may then be filtered, which may allow for estimation of the frequency distance, or average frequency distance, between adjacent peaks in the spectrum, or at least a portion of the spectrum/data set.

The frequency distance between adjacent peaks in the data set may then be used to calculate an estimated breathing rate or another vital sign for all of the range bins or other data collections of interest. In some embodiments, the vital sign may be calculated/estimated by calculating an average/mean, weighted mean, or median distance between adjacent Doppler spectrum peaks in each of the range bins/data collections of interest, in some cases of a predetermined period of time.

Each of the various breathing rates or other vital signs of each of the bins or other data collections may, in some embodiments, be combined into a single, current vital sign. Again, this may be accomplished by using the target range bin/data collection, or the target range bin/data collection and a certain number of adjacent or otherwise sufficiently related range bins/data collections, as previously mentioned. The rate associated with the current vital sign may be processed, for example, as a rolling average/mean, weighted mean, or median over a predetermined time period.

In some embodiments and implementations, the breathing rate or other vital sign may be filtered before being transmitted to another module and/or component of the vehicle. This may be accomplished, for example, using a smoothing filter, such as an alpha-filter, a Kalman filter, or the like. The resulting vital sign data may then be used to take a variety of actions and/or parameter changes, as described in greater detail below.

Some embodiments may be configured to process joint features, which may combine, for example, any of the features referenced herein to allow a joint distribution to be used. In some cases, this joint distribution may be used during occupant/object classification. For example, in some embodiments and implementations, range data, such as minimum range values for valid detections, may be combined with Doppler spectrum data and/or number of detections, to provide one or more joint features to be used in further processing or other steps, such as occupant classification. Thus, it should be understood that any of the features calculated or otherwise used, as indicated in elements 638-648, for example, may comprise a joint feature, which may include, for example, any two of the features described herein. Of course, in some embodiments, additional features may be used (or fewer features) beyond the six shown in FIG. 6C, any one of which may comprise a joint feature, may be calculated and/or used.

Data from feature extraction 632 may then, in some embodiments, be used for occupant classification at 634. In some embodiments, occupant classification may comprise calculation of various statistical models, parameters, and/or related statistics using and/or otherwise relating to one or more of the various features from the feature extraction 632. The statistical calculations may be made over time, as indicated at 650. In some embodiments, one or more ratios may be calculated using one or more of the aforementioned features.

For example, ratios involving the number of detections, the spread bandwidth, the range extent, the minimum and/or maximum range, the variance of the frequency difference between Doppler peaks, vital sign estimate data ratios, and/or ratios of joint features may be used. Exemplary ratios that may be useful in connection with various embodiments may be calculated as follows:

$${ratio\_num}_{i} = \frac{n u {m}_{i}}{{\sum }_{i} n u {m}_{i}}$$

where numᵢ is the number of detections for the i^{th} class;

$${ratio\_spect}_{i} = \frac{S {B}_{i}}{{\sum }_{i} S {B}_{i}}$$

where SBᵢ is the spread bandwidth for the i^{th} class;

$${ratio\_range}_{i} = \frac{{num\_range}_{i}}{{\sum }_{i} {num\_range}_{i}}$$

where num_rangeᵢ is the range extent for the i^{th} class;

$${ratio\_range}_{-} {min}_{i} = \frac{num\_range {\_min}_{i}}{{\sum }_{i} num\_range {\_min}_{i}}$$

where num_range_minᵢ is the minimum range for the i^{th} class;

$$ratio\_freq {\_var}_{i} = \frac{{FD\_var}_{i}}{{\sum }_{i} {FD\_var}_{i}}$$

where FD_varᵢ is the variance of the frequency-difference between doppler peaks for the i^{th} class;

$${ratio\_vital}_{i} = \frac{{vital}_{i}}{{\sum }_{i} {vital}_{i}}$$

where vitalᵢ is the estimate of the vital sign for the i^{th} class;

$${ratio\_joint}_{i} = \frac{{joint}_{i}}{{\sum }_{i} j o i n {t}_{i}}$$

where jointᵢ is the joint estimate may comprise the minimum range and spectrum, or the number of detections and spectrum peaks.

After calculating various statistics and/or statistical parameters, an initial classification may be made at 652. For example, one or more occupants in the vehicle may be classified according to a predicted age group, such as adult/senior/child/infant, a location and/or status of the occupant within the vehicle, such as driver, etc. In some embodiments, the resulting statistical features may be processed through a decision tree to determine whether the class is being identified for the current time.

Some embodiments may comprise filtering the classification decision over time, as indicated at 654. For example, some embodiments may comprise increasing a counter for one or more classes if/when a class is identified. Similarly, some embodiments may comprise, either additionally or alternatively, decreasing the counter if/when a class is not identified for the current time. Some embodiments may comprise selecting an output class for the class with the highest counter, in some cases the highest counter beyond a predetermined threshold.

Some embodiments may comprise measurement of a parameter indicative of the quality of the classification decision, as indicated at 656. For example, in some embodiments, this may comprise use of the class count, which may indicate the firmness and/or quality of the classification decision.

The various data from one or more of these processing steps may be sent to the classifier output 658, which may then be used in various other ways within the vehicle, such as enabling or disabling airbag cushions, changing sensitivity levels for future data processing, changing operational parameters of the vehicle, etc. By comparing FIGS. 6A and 6B with FIG. 6C, it can also be seen that the classifier output may be reset or otherwise changed based upon scene change data as well.

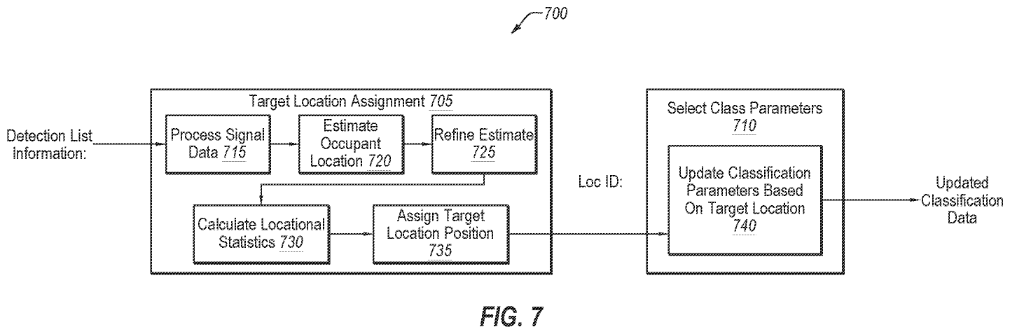

FIG. 7 illustrates an example of a system 700 for location assignment of a vehicle occupant or other target within a vehicle and/or classification using such location assignment(s) according to some embodiments. As can be appreciated from comparing FIG. 7 with FIGS. 6A-6C, any of the sub-elements, components, and/or steps of any of the embodiments and/or implementations disclosed herein, including but not limited to that of FIGS. 6A-6C, may be separated out into individual systems, used to replace components, elements, steps with other components/elements/steps, removed from a system, or otherwise modified as those of ordinary skill in the art would appreciate after having gained the benefit of this disclosure.

Thus, signal data from a RADAR sensor or other electromagnetic sensor, such as a detection list data set, may be fed into system 700 and processed, in some embodiments as part of a target location assignment module or system 705, at 715. As previously mentioned, in some embodiments, this initial processing step may comprise selection of a subset of data, such as a selection of one or more valid signal detections, from the incoming data set. As a more specific example, in some embodiments, processing of signal data at 715 may comprise identifying signals with a maximum and/or threshold SNR. In some embodiments, additional detections that are sufficiently close to the maximum SNR detection and/or the range of the detections having the maximum and/or threshold SNR may also be selected for further location processing.

An estimate of a location of one or more vehicle occupants may then be made at 720. As previously mentioned, this estimate may be made to assign each target/occupant with a particular seat in the vehicle. Alternatively, the estimate may be less refined, and may, for example, assign a target to a particular row, to a front/back of the vehicle, and/or left/right lateral side of the vehicle. In some embodiments, the target location may be estimated using, for example an SNR weighted average of one or more dimensional coordinates of each of the selected detections, such as the “X” and “Y” coordinates, which may correspond to the front/rear and lateral side to side coordinates of the vehicle.

In some embodiments, the estimate made at 720 may be refined at 725. In some embodiments, this refinement may comprise a compensation step involving one or more of the variables/coordinates of the data set, such as the X coordinate. This compensation may, for example, be based upon whether a significant number, such as a threshold number and/or percentage, of the X-coordinate (or Y-coordinate) values fall within a particular side/range/threshold, such as in the front side/rear side of the vehicle, to avoid dithering of the location assignment.

Statistics based upon the locational data obtained may then be calculated at 730. For example, in some embodiments, calculation of locational statistics may comprise calculation of one or more detection ratios, such as front footwell, front, and/or rear detection ratios. Other statistical calculations and/or data may be used in finalizing the target location position, such as range data, range extent data, and the like. In some cases, data from other sensors, such as weight sensors, temperature sensors, audible sensors, and the like, may also be used in either the initial estimation, refinement, and/or statistical calculation steps of the system.

The processed location data may then be used to assign the target to a particular seat, location, and/or zone at 735. For example, in some embodiments, the ratio(s) or other statistical data calculated at 730 may be used to assign the target to a particular row of the vehicle. In some embodiments, this row assignment may be combined with similar data used to assign the target's position further, such as in terms of left to right lateral position and/or to a particular seat in the vehicle. This target location assignment data may then be saved and used for further actions/processing.

The location ID data may then be fed to other modules of system 700 and/or to other systems or sub-systems within a vehicle. For example, in the depicted embodiment, the location ID data for one or more targets within the vehicle may be used to select classification parameters at 710.

In some such embodiments, classification parameters may be assigned, or updated, at 740 based upon the target location assignments received from the target location assignment module 705. In some embodiments, for one or more locations within the vehicle, in some cases each location that could correspond with a distinct occupant within the vehicle, parameters that define the range (upper and lower bounds) or a threshold of one or more feature values for each location may be used to determine whether estimated values for these features fall within predetermined threshold and/or range regions. Updated classification data, such as data corresponding with these threshold and/or range regions, may then be output and used for further processing and/or actions.

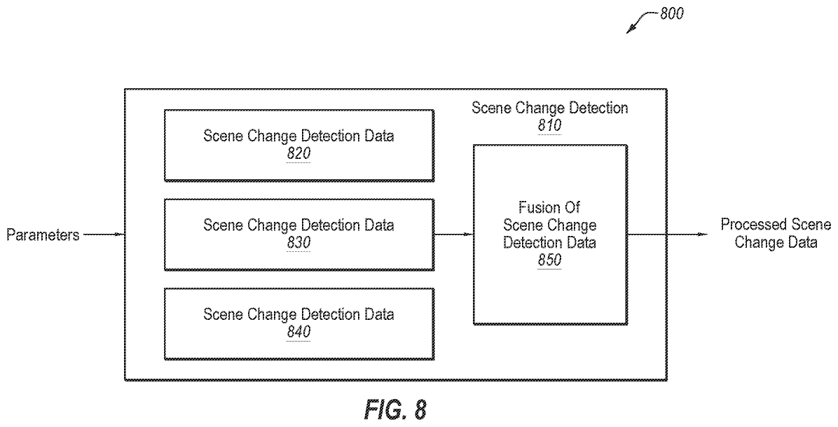

FIG. 8 depicts an example of a system 800 for detection and/or processing of scene change data within a vehicle using, at least in part, RADAR or other electromagnetic signals. Parameters may be received within a scene change detection module 810, such as, for example, parameters relating to signal detections, vital signs, and/or target location data.

In some embodiments, incoming data may alternatively, or further comprise messages and/or data from other systems in the vehicle, such as data indicating that the vehicle has been turned on, turned off, and/or one or more doors of the vehicle have been opened and/or closed. Various types of scene change detection data, as indicated as 820, 830, and 840, may, in some embodiments, be fused or otherwise combined, as indicated at 850, in order to detect a scene change. For example, some embodiments may be configured to use scene change detection data comprising signal detection-based scene change data, door and/or vehicle message-based scene change data, and vehicle speed/velocity-based scene change data.

As previously mentioned, signal detection-based scene change data may comprise, for example, a threshold number or percentage change in the number of detections associated with the vehicle, or a particular part of the vehicle. In some embodiments, the types of detections and/or the values for certain detections in the vehicle, or a particular part of the vehicle, may be used to indicate of a scene change. Such data may also, in some embodiments, comprise locational data and/or vital sign data, which may be used to detect threshold movement in a cabin.

As another type of scene change detection data, some embodiments may comprise use of data from sensors associated with one or more doors of the vehicle, or other sensors indicating that a change has occurred within the vehicle that might warrant setting of parameters or other data, heightened signal processing sensitivity, or lessened or entirely omitted signal processing data in certain regions of the vehicle.

As another example of a possible type of scene change detection data, some embodiments may be configured to use vehicle speed/velocity-based data. For example, when the vehicle speed changes beyond a certain threshold and/or beyond a certain period of time, data may be used by scene change detection module 810 to result in a change in how vehicle occupant locations, vital signs, and/or classifications are processed or otherwise used.

Yet another example of scene change detection data may comprise data from other sensors, including sensors not involving use of electromagnetic radiation, such as pressure, force, and temperature sensors, for example, which may indicate the presence, movement, or lack of presence of a vehicle occupant within a particular seat.

The resulting processed scene change data may then be used in various ways by, for example, other modules and/or systems of the vehicle. For example, in some embodiments, a decision may be made using the processed scene change data as to whether a scene change, or at least a scene change of significant relevance, has likely taken place. In some embodiments, certain types of scene change data, such as data indicative of a door opening and/or closing or data indicative of the addition or subtraction of a vehicle occupant from the vehicle or one or more seats within the vehicle, for example, may automatically trigger a scene change. However, other types of scene change data may require processing and/or analysis to determine whether a scene change has taken place. For example, confirmation of a scene change may require the confluence of speed change data, such as speed change beyond a threshold or any change to or from zero, with time data, or a sufficient change between current RADAR detection data and previous comparative data, prior to confirmation of a scene change worthy of further consideration and/or processing within system 800.

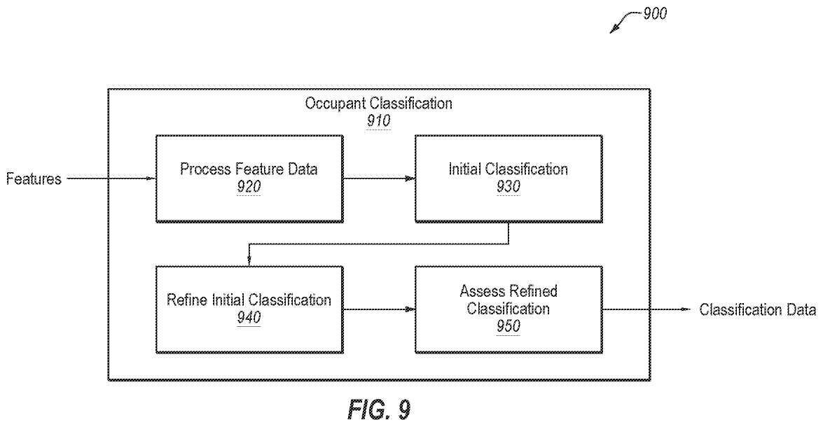

An example of a system 900 for occupant classification is depicted in FIG. 9. Again, in some embodiments, system 900 may comprise a module of another system. Various features, which may include any of the various features mentioned herein and derivatives thereof, that may be used in occupant or another object classification module/system 910 are processed at 920. In some embodiments, this processing may comprise calculating or otherwise processing features by creating statistical parameters or other statistical elements of one or more features.

For example, in some embodiments, one or more statistical ratios may be calculated using one or more of the aforementioned features, such as ratios involving the number of detections, the spread bandwidth, the range extent, the minimum and/or maximum range, the variance of the frequency difference between Doppler peaks, vital sign estimate data ratios, and/or ratios of joint features involving combining two or more features.

An initial classification may then be made at 930 using the processed feature data. For example, a particular occupant within a particular seat and/or region of the vehicle may be classified according to their estimated age/age range, location, and/or arousal level. The arousal level may be used, for example, to assess whether an occupant is awake and may comprise comparison between a baseline and/or expected vital sign and a current vital sign.

Initial classifications may be refined at 940. In some embodiments, this refinement may comprise filtering the classification decision(s) over time. For example, some embodiments may comprise increasing a counter for one or more classes if/when a classification has been made. Similarly, some embodiments may comprise, either additionally or alternatively, decreasing the counter if/when a classification is not made for the current time. Some embodiments may comprise selecting an output class for the class with the highest counter, in some cases the highest counter beyond a predetermined threshold.

Some embodiments may comprise assessing the refined classification at 950. For example, some embodiments may comprise measurement of a parameter indicative of the quality of the classification decision, as previously mentioned. For example, in some embodiments, this may comprise use of the class count, which may indicate the firmness and/or quality of the classification decision.

The resulting classification data from one or more of these processing steps may be sent to another module and/or system. As mentioned throughout this disclosure, such data may, for example, be used in various other ways within the vehicle, such as enabling or disabling airbag cushions, changing sensitivity levels for future data processing, changing operational parameters of the vehicle, providing notices to current vehicle occupants and/or an owner/user/previous occupant of the vehicle, and the like.

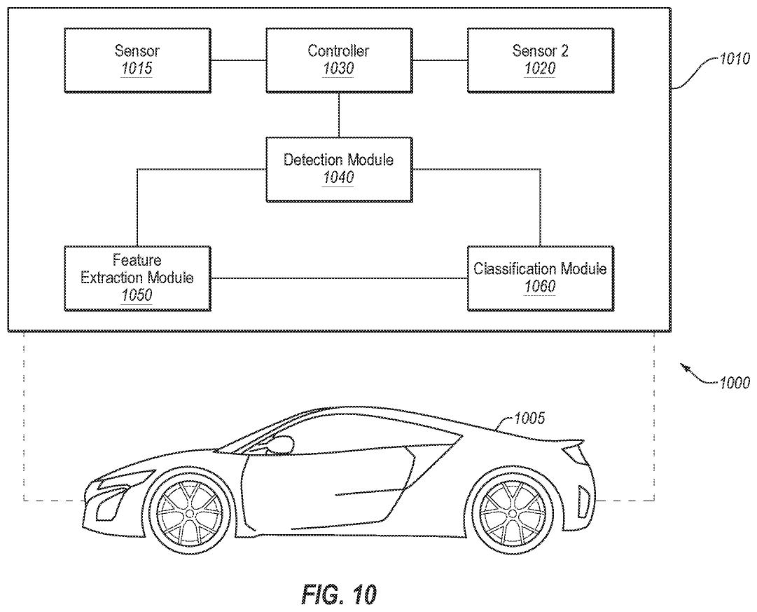

FIG. 10 illustrates an example of a system 1000 for processing of vehicle occupant features and/or classification of occupants in a vehicle 1005. System 1000 may comprise an internal system 1010, which may combine a combination of various hardware, software, firmware, and the like as desired. System 1000 comprises a first sensor module 1015 and a second sensor module 1020. Those of ordinary skill in the art will appreciate that, although two sensors/sensor modules are shown in the depicted embodiment, the number of sensors may vary as desired without departing from the primary inventive principles of the system 1010, including a single sensor or more than two sensors.

First sensor module 1015 and second sensor module 1020 may comprise any number of sensors, such as RADAR sensors, LIDAR sensors, or other sensors configured to send and/or receive electromagnetic radiation, as desired. Although it may be preferred to have at least one RADAR or other electromagnetic sensor, some embodiments may comprise other sensors not involving use of electromagnetic radiation, such as pressure, force, temperature, or other sensors, the data from which may be combined with RADAR or other electromagnetic radiation sensors as described throughout this disclosure. Sensor modules 1015 and 1020 may further comprise various other software, hardware, and/or firmware elements as desired in order to send and receive signals for processing by other modules.

System 1010 further comprises a controller 1030, which may be configured to process data from sensor modules 1015/1020. As used herein, the term “controller” refers to a hardware device that includes a processor and preferably also includes a memory element. The memory may be configured to store one or more of the modules referred to herein and the controller 1030 and/or one or more processors may be configured to execute the modules to perform one or more processes described herein.

System 1010 further comprises a detection module 1040 that is coupled with both of the sensor modules 1015/1020. Of course, in some embodiments, a separate detection module may be provided for each sensor and/or sensor module, if desired. Detection module 1040 may be configured to receive raw, sensed data from the sensors of sensor modules 1015/1020 and attempt to identify/detect occupants within vehicle 1005 using such data, such as by detecting evidence of breathing or another vital sign, as described above and throughout this disclosure. Although in preferred embodiments system 1010 may be configured specifically to detect human occupants, it is contemplated that the principles herein may also be used to detect other living occupants of a vehicle, such as dogs, cats, or other pets. Some embodiments may also be configured to detect certain objects within a vehicle, either together or independent from whether living objects are present, such as the presence of an infant car seat, for example.

Detection module 1040 may be communicatively coupled with a feature extraction module 1050. Feature extraction module 650 may be configured to process incoming data so as to identify, for example, a vital sign, such as a breathing rate or heart rate, of an occupant and estimate the rate, as described above and throughout this disclosure. The resulting vital sign may then be used by system 1010 to modify one or more features/parameters of vehicle 1005 and/or to otherwise take actions based upon such data, as also described throughout this disclosure.

Of course, other types of features may also be extracted and/or processed by feature extraction module 1050, including features relating to and/or from which rates associated with vital signs may be derived and other features that need not related to such vital signs. For example, features may comprise variance feature data, such as variance of frequency-difference between Doppler peaks, spread bandwidth features, features comprising the number of useful/valid detections from a RADAR sensor, features comprising the number of valid Doppler spectrum peaks, range feature data, range extent feature data, and other features from which locational data may be derived.

In some embodiments, this data may be used by a classification module 1060 to classify the vehicle occupant associated with a particular estimated vital sign, a location, or otherwise. Of course, in some embodiments, classification module 1060 may be configured to classify each occupant within vehicle 1005 based upon a separate vital sign and/or locational data associated with each vehicle occupant. As an example of a useful classification based at least partially on a breathing rate or other vital sign, classification module 1060 may be configured to classify the occupant(s) as an infant, child, adult, and/or senior citizen. In some embodiments, for example, classification module 1060 may be configured with one or more predetermined ranges of breathing rates or other breathing rates based upon statistical data correlating age with such vital sign(s), such as the data depicted in FIG. 4. Some embodiments may also, or alternatively, be configured to classify vehicle occupants according to their state of awareness/arousal, which may be based at least partially on vital sign data.

In some embodiments, classification module 1060 may be configured to use a statistical analysis of the incoming vital sign data alone to classify the occupant(s). Alternatively, other parameters and/or features may be used in conjunction with the parameter/feature derived from the statistical analysis to classify occupants, such as data indicative of a size/weight of occupants, which could also be derived from the same RADAR sensor or other electromagnetic radiation data. Alternatively, such data may be obtained from other sensors, such as weight sensors, temperature sensors, cameras, and the like. Thus, it should be understood that the term sensor in, for example, FIG. 10, should be considered to encompass, in some contemplated embodiments, such other sensors. However, it should also be understood that, in some preferred embodiments, this term may be limited to electromagnetic sensors, such as RADAR sensors.

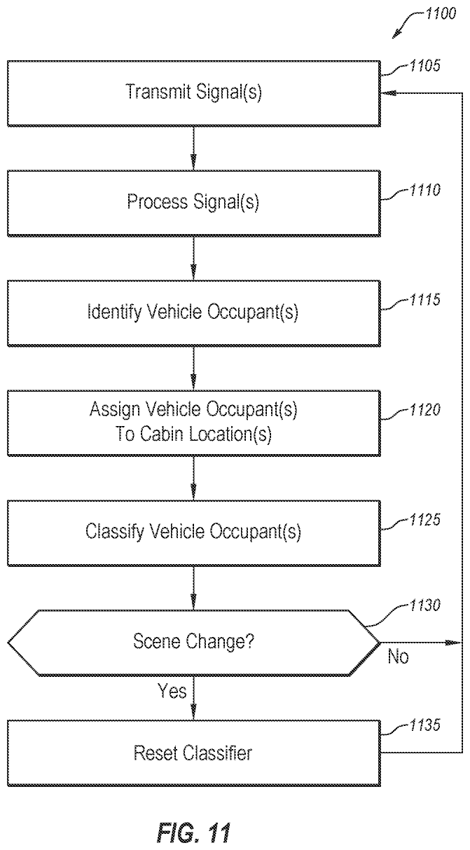

FIG. 11 is a flow chart depicting an example of a method 1100 for classification of vehicle occupants according to some implementations. Method 1100 may begin with the transmission 1105 of various electromagnetic signals, such as RADAR signals, from one or more sensors, as described above. These signals may, in some implementations, be directed to specific locations corresponding with seats of the vehicle, may be beamformed using multiple sensors, or may be distributed more widely throughout the vehicle so as to detect possible occupants located elsewhere in the vehicle. Also, a single sensor may be used to transmit signals to a plurality of seats/locations or multiple sensors may be used as desired. For example, a separate sensor may be used for each seat, a separate sensor may be used for each row of the vehicle, or one or more sensors may be positioned along a central portion of the vehicle from one lateral side of the vehicle to the other, as shown in FIG. 1.

Signals may then be received and/or processed at 1110. In some implementations, these signals may comprise reflected signals, but this need not be the case for all contemplated implementations. Rather, in some implementations, step 1110 may comprise receiving a signal at a second sensor sent from a first sensor.

Locations of one or more vehicle occupants may then be identified at 1115. As mentioned above, this step may comprise use of electromagnetic sensors and/or vital sign data, or need not involve one or both of these features. For example, RADAR or other electromagnetic sensors may be used to obtain range, range extent or other data from which the presence of an object, living or otherwise, may be derived without explicitly using vital sign data. Similarly, other types of sensors may be used in other embodiments to detect the presence, such as a human vehicle occupant in preferred embodiments, within the vehicle cabin.

The location of each vehicle occupant identified in step 1115 may then be identified and/or assigned to a particular seat or other location in the vehicle at 1120. This step/process may, for example, involve one or more of the steps, modules, and/or elements previously discussed in connection with FIGS. 6A-6C and/or 7.

Once a seat, location, and/or zone has been identified with a particular occupant, or each occupant in a vehicle, one or more of the vehicle occupants may be classified at 1125. This classification may involve use of one or more of the features described throughout this disclosure, such as, for example, vital sign data and/or vital signs derived and/or estimated from such data. For example, a vital sign may be associated with a repeating signal pattern and/or a particular occupant and then the distance between adjacent peaks in the pattern, or a statistical analysis of such pattern(s), such as interpolation, mean, weighted mean, and/or median, for example, of a distance between adjacent peaks and/or an initial vital sign estimate, may be used to determine and/or refine the vital sign estimate. In some implementations, additional processing steps, such as applying smoothing filters of the vital sign estimate, may also take place. Preferably, this vital sign estimate is then processed and refined over time to maintain a real time, or at least substantially real time estimation of the vital sign of one or more occupants in the vehicle.

In some implementations, vehicle occupants may be classified based upon their predicted age group, which may be based wholly, or at least partially, on the vital sign estimate and/or other data, such as the occupant location data. This may involve use of vital sign thresholds and/or vital sign ranges. For example, if a detected breathing rate is at least 30 breaths per minute, the associated vehicle occupant may be classified as a child. In some implementations, the classification may require a stable rate detection, such as an estimation within the threshold and/or range over a predetermined period of time, so as to prevent temporary increases in breathing rate or another vital sign from re-classifying an occupant.

Occupants may also be classified based upon a threshold associated with old, rather than young, age, and/or health conditions that may be associated with a particular vital sign. For example, if a sufficiently low, and preferably stable, breathing rate or other vital sign is detected, an occupant may be classified as a senior.

As another example of a possible classification based at least partially on a vital sign estimated using RADAR or another electromagnetic wave signal, in some implementations and embodiments, occupants may be classified based upon a detected change in a vital sign. For example, if a breathing rate, heart rate, or another detected vital sign drops by a predetermined amount, such a predetermined percentage or predetermined raw number of breaths/beats per minute in a relatively simple example, the occupant may be classified as sleeping or otherwise having a noteworthy condition. In some implementations and embodiments, the method/system may be configured to make this classification only for the driver, since other occupants sleeping may not be of concern. Some implementations and embodiments may also, or alternatively, be configured to detect threshold increases in vital signs over time, which may be used to classify an occupant as having a panic attack or another noteworthy condition.

Some vehicles/systems/methods may then be configured to take automated actions based upon vehicle occupant classifications and/or re-classifications. For example, some vehicles may be configured to automatically disable airbags associated with a seat in which an occupant has been classified as a child/infant and/or in which no occupant vital sign can be detected.

Similarly, some embodiments and implementations may be configured to monitor the presence, or lack thereof, of an occupant and take one or more actions based at least partially thereon, which may be considered within the scope of various contemplated implementations of method 1100. For example, after classifying an occupant as a child or infant, upon detecting that the vehicle has been stopped, turned off, and/or the driver and/or other occupants have exited a vehicle, in some cases after a threshold period of time, the vehicle may be configured to send a warning/notification, turn on an air conditioning unit or heater, and/or notify relevant authorities upon detecting that a child has been left in the vehicle.

In some cases, this warning/notification/action may only take place upon detecting other conditions, such as a sufficiently high, or low temperature. Similarly, in some cases, the warning/notification/action may only take place after a sufficiently long period of time has elapsed since the child has been left. This time period, however, may be reduced or eliminated depending upon the estimated age of the occupant, the temperature, and/or other conditions. The time, temperature, and/or other conditions needed to trigger a warning/notification/action may scale with the projected age of the occupant. For example, the threshold time and/or temperature from room temperature needed to trigger a warning/notification/action may decrease as the projected age of the occupant decreases.

As another example, some vehicles/systems/methods may be configured to automatically take an action based upon a detected change in vital sign, such as a sufficiently dramatic increase or decrease in the vital sign. Such action(s) may comprise, for example, triggering a warning/notification/action, either within the vehicle or to a device remote from the vehicle, such as a smartphone. In some embodiments, if a dramatic increase or decrease in the vital sign, or another vital sign condition that is indicative of danger, is detected, the vehicle may be configured to take control from the driver (or, in the case of an autonomous vehicle, simply reconfigure a current driving instruction set) to slow the vehicle, pull the vehicle to the side of the road, and/or stop the vehicle.

As another example, in some embodiments and implementations, if a vital sign or vital sign change is indicative of a problematic condition, such as an elderly occupant or one or more health conditions, the vehicle may be configured to enhance monitoring of the vital sign by, for example, tuning the RADAR to more specifically and/or more closely monitor the vital sign and/or other vital signs of the vehicle occupant associated with the problematic vital sign. In some cases, the vehicle may be configured to, additionally or alternatively, target other monitoring systems, sensors, or the like for the particular occupant of concern.

For example, to assist in monitoring a possible health condition or other condition concerning for the safety of the vehicle occupants, detection of a particular vital sign or vital sign change associated with a particular occupant (again, the driver of the vehicle may warrant more attention and therefore less stringent requirements to trigger a warning/action than other occupants) may trigger actuation of another sensor and/or monitor, such as a camera, within the vehicle.

Such processing and/or actions may take place at any suitable point throughout method 1100.

Data indicative of a relevant change of scene within the vehicle may also be used in method 1100, as described throughout this disclosure. This data may be processed (again, as described throughout) in order to determine whether a relevant scene change has taken place at 1130. In the event of a relevant scene change, or the determination that a relevant scene change is likely, method 1100 may proceed to 1135 at which point the classifier, at least in part, may be reset. In some embodiments, one or more parameters and/or buffers may be reset, which may indicate that previous data associated with, for example, a particular seat or seats, may no longer be useful/reliable. In the event that the scene change data is absent or fails to indicate the presence or likely presence of a relevant scene change, method 1100 may revert to an earlier step to resume collection and/or processing of data without resetting the classifier, or at least a portion thereof.

As another example, in some embodiments and implementations, a vehicle may be configured to detect the presence of an unexpected occupant, such as a vital sign where none would be expected and/or an unexpected scene change, for security reasons. For example, if the vehicle has been turned off, shut down, or locked without a subsequent unlocking and/or proper re-starting of the vehicle, or if a scene change has been detected without an expected event correlated therewith, such as opening and/or entering of the vehicle without a suitable key or other security feature, the vehicle may be configured to monitor for vital signs and/or scene changes and, upon detection of a vital sign and/or scene change under these or other circumstances under which a vital sign and/or scene change would not be expected, the vehicle and/or an associated application and/or system may be configured to report the incident to the owner of the vehicle and/or the police or another suitable authority to provide enhanced security.

As another example, in some embodiments and implementations, a vehicle may be configured to provide pre-collision protection and/or post-collision assistance based, at least in part, on vital sign detection/data, scene change data, and/or classification data. For example, the vehicle may be configured to adjust certain collision safety features, such as airbags, based upon the size, age, and/or presence of a particular occupant within a particular seat of the vehicle. Similarly, the vehicle may be configured to obtain, store, and/or use data about the presence and/or health of vehicle occupants using vital sign data, such as comparing vital sign data prior to a collision with vital sign data following a collision, which may allow the vehicle to assess whether all of the occupants within the vehicle are still present in the vehicle following a collision and/or assess the current health of the vehicle occupants by comparing pre- and post-collision vital signs, for example, in some cases along with other data, such as data from other sensors and/or scene change data.

As used herein, a software module or component may include any type of computer instruction or computer executable code located within a memory device and/or m-readable storage medium. A software module may, for instance, comprise one or more physical or logical blocks of computer instructions, which may be organized as a routine, program, object, component, data structure, etc., that perform one or more tasks or implements particular abstract data types.

In certain embodiments, a particular software module may comprise disparate instructions stored in different locations of a memory device, which together implement the described functionality of the module. Indeed, a module may comprise a single instruction or many instructions, and may be distributed over several different code segments, among different programs, and across several memory devices. Some embodiments may be practiced in a distributed computing environment where tasks are performed by a remote processing device linked through a communications network. In a distributed computing environment, software modules may be located in local and/or remote memory storage devices. In addition, data being tied or rendered together in a database record may be resident in the same memory device, or across several memory devices, and may be linked together in fields of a record in a database across a network.

Furthermore, embodiments and implementations of the inventions disclosed herein may include various steps, which may be embodied in machine-executable instructions to be executed by a general-purpose or special-purpose computer (or other electronic device). Alternatively, the steps may be performed by hardware components that include specific logic for performing the steps, or by a combination of hardware, software, and/or firmware.

Embodiments and/or implementations may also be provided as a computer program product including a machine-readable storage medium having stored instructions thereon that may be used to program a computer (or other electronic device) to perform processes described herein. The machine-readable storage medium may include, but is not limited to: hard drives, floppy diskettes, optical disks, CD-ROMs, DVD-ROMs, ROMs, RAMS, EPROMS, EEPROMs, magnetic or optical cards, solid-state memory devices, or other types of medium/machine-readable medium suitable for storing electronic instructions. Memory and/or datastores may also be provided, which may comprise, in some cases, non-transitory machine-readable storage media containing executable program instructions configured for execution by a processor, controller/control unit, or the like.

The foregoing specification has been described with reference to various embodiments and implementations. However, one of ordinary skill in the art will appreciate that various modifications and changes can be made without departing from the scope of the present disclosure. For example, various operational steps, as well as components for carrying out operational steps, may be implemented in various ways depending upon the particular application or in consideration of any number of cost functions associated with the operation of the system. Accordingly, any one or more of the steps may be deleted, modified, or combined with other steps. Further, this disclosure is to be regarded in an illustrative rather than a restrictive sense, and all such modifications are intended to be included within the scope thereof. Likewise, benefits, other advantages, and solutions to problems have been described above with regard to various embodiments. However, benefits, advantages, solutions to problems, and any element(s) that may cause any benefit, advantage, or solution to occur or become more pronounced, are not to be construed as a critical, a required, or an essential feature or element.

Those having skill in the art will appreciate that many changes may be made to the details of the above-described embodiments without departing from the underlying principles of the invention. The scope of the present inventions should, therefore, be determined only by the following claims.

1. A method for identification of a vehicle occupant within a vehicle cabin, the method comprising the steps of:
identifying a vehicle occupant within a vehicle using electromagnetic signals;
assigning the vehicle occupant to a location within the vehicle by processing electromagnetic signals; and
extracting one or more features about the vehicle occupant by processing electromagnetic signals.

2. The method of claim 1, classifying the vehicle occupant by processing electromagnetic signals

3. The method of claim 2, wherein the step of identifying a vehicle occupant comprises detecting a vital sign of the vehicle occupant.

4. The method of claim 3, wherein the vital sign comprises a breathing rate of the vehicle occupant.

5. The method of claim 4, wherein the breathing rate is calculated by:
identifying a repeating pattern of Doppler spectrum peaks in a RADAR signal using at least one range bin;
identifying an estimated frequency distance between adjacent peaks of the repeating pattern; and
calculating an estimated breathing rate using the estimated frequency distance.

6. The method of claim 1, further comprising identifying a scene change within the vehicle.

7. The method of claim 6, wherein the scene change comprises at least one of:
identifying a change in a number of vehicle occupants within the vehicle using electromagnetic signals;
identifying a change in a location of vehicle occupants within the vehicle using electromagnetic signals;
identifying movement in a door of the vehicle; and
identifying a threshold change in velocity of the vehicle.

8. The method of claim 7, further comprising classifying the vehicle occupant by processing electromagnetic signals.

9. The method of claim 8, further comprising, in response to identifying the scene change, changing at least one processing parameter associated with at least one of the steps of extracting one or more features about the vehicle occupant and classifying the vehicle occupant.

10. A method for classification of an object within a vehicle using RADAR signal processing, the method comprising the steps of:
identifying an object within a vehicle using RADAR signals;
assigning the object to a location within the vehicle by processing RADAR signals;
extracting one or more features about the object by processing RADAR signals;
identifying a scene change within the vehicle; and
in response to identifying the scene change, changing at least one processing parameter of the vehicle.

11. The method of claim 10, further comprising classifying the object by processing RADAR signals.

12. The method of claim 10, wherein the object comprises a human, and wherein the step of extracting one or more features about the object by processing RADAR signals comprises estimating a rate associated with a vital sign of the human within the vehicle.

13. The method of claim 12, wherein the step of extracting one or more features about the object by processing RADAR signals comprises calculating a variance of a frequency difference between Doppler peaks associated with the RADAR signals.

14. The method of claim 12, wherein the object comprises an occupant of the vehicle, and wherein the step of extracting one or more features about the object by processing RADAR signals comprises:
identifying a repeating pattern of Doppler spectrum peaks in a RADAR signal using one or more range bins;
identifying an estimated frequency distance between adjacent peaks of the repeating pattern; and
calculating an estimated rate of a repeating vital sign of the occupant within a cabin of the vehicle using the estimated frequency distance.

15. A system for classification of a vehicle occupant using electromagnetic signal processing, comprising:
an electromagnetic sensor positioned within a cabin of a vehicle;
a location detection module configured to process reflected electromagnetic signals to estimate a location of a vehicle occupant within the cabin of the vehicle; and
a feature extraction module configured to extract one or more features about the vehicle occupant by processing reflected electromagnetic signals.

16. The system of claim 15, wherein the feature extraction module is configured to identify a vital sign associated with the vehicle occupant.

17. The system of claim 16, wherein the feature extraction module is configured to use a Doppler signal repetition frequency to estimate a rate associated with a vital sign of the vehicle occupant.

18. The system of claim 17, wherein the rate comprises at least one of a breathing rate and a heart rate.

19. The system of claim 15, wherein the electromagnetic sensor comprises a RADAR sensor.

20. The system of claim 15, further comprising a classification module configured to classify the vehicle occupant using features extracted using the feature extraction module, wherein the classification module is configured to classify the vehicle occupant according to an estimated age group using a rate associated with a vital sign of the vehicle occupant obtained from the electromagnetic sensor.
